(19) United States
(12) Patent Application Publication
(10) Pub. No.: US 2021/0046300 A1
(43) Pub. Date: Feb. 18, 2021

(54) HOSE CADDY

(71) Applicant: Leonard Lawrence Peterson, Hugo, MN (US)

(72) Inventor: Leonard Lawrence Peterson, Hugo, MN (US)

(21) Appl. No.: 16/539,032

(22) Filed: Aug. 13, 2019

(51) Int. Cl. A61M 39/08; B65H 75/36

(52) U.S. Cl. CPC A61M 39/08; B65H 2701/33; B65H 75/362

(57) ABSTRACT

A hose caddy includes a body made of a rigid material. The body includes a tube shape with an outer surface and an inner surface. The inner surface defines a channel having a first open end, and a second open end. One, or more, couplers protrude from a rear side of the outer surface of the body and couples the body to a surface. At least one hook protrudes from a front side of the outer surface of the body.

### BACKGROUND OF THE INVENTION

The present invention relates to containing hoses and, more particularly, to a hose caddy for containing hoses.

Medical devices, such as bilevel positive airway pressure (BiPAP) and continuous positive airway pressure (CPAP), have hoses carrying air, fluids and other elements required for delivery to the patient. The hoses often get in the way and are tangled by the patient or care provider. Additionally, the hoses can be damaged and disconnected by accident after movement by the patient or care provider. The hoses may be expensive and can be hazardous to the patient and other persons visiting the bedside when restricted or damaged.

As can be seen, there is a need for a hose caddy for containing hoses.

### SUMMARY OF THE INVENTION

In one aspect of the present invention, a hose caddy comprises: a body made of a rigid material and comprising a tube shape with an outer surface and an inner surface, the inner surface defining a channel having a first open end, and a second open end; a coupler protruding from a rear side of the outer surface of the body and configured to couple the body to a surface; and at least one hook protruding from a front side of the outer surface of the body.

In another aspect of the present invention, a method of containing a hose comprises: providing a hose caddy comprising a body made of a rigid material and comprising a tube shape with an outer surface and an inner surface, the inner surface defining a channel having a first open end, and a second open end; coupling the Caddy body to a surface above a patient rest area; and running the hose from a device through the channel of the hose caddy, then to a patient.

These and other features, aspects and advantages of the present invention will become better understood with reference to the following drawings, description and claims.

### BRIEF DESCRIPTION OF THE DRAWINGS

FIG. 1 is a perspective view of an embodiment of the present invention, shown in use;

FIG. 2 is a perspective view of an embodiment of the present invention, shown in use;

FIG. 3 is a section view of an embodiment of the present invention, taken along line 3-3 in FIG. 1;

FIG. 4 is an exploded view of an embodiment of the present invention; and

FIG. 5 is a flow chart of an embodiment of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

The present invention includes a tubular guide for supporting hoses in a position that is out of harm's way. The present invention may be coupled to a clean and smooth surface away from the patient on a bed headboard or on a point of the room wall. The air and/or fluid lines are threaded through the guide to minimize interference by movement of the patient or care provider and from contacting these lines by accident or normal movement.

In addition, the guide of the present invention may provide the patient and/or care provider a hanger to rest air or fluid masks and lines, keeping them out of the path of normal movements, when not in active use. The present invention may be easily moved to another location without damage to the mounting surface.

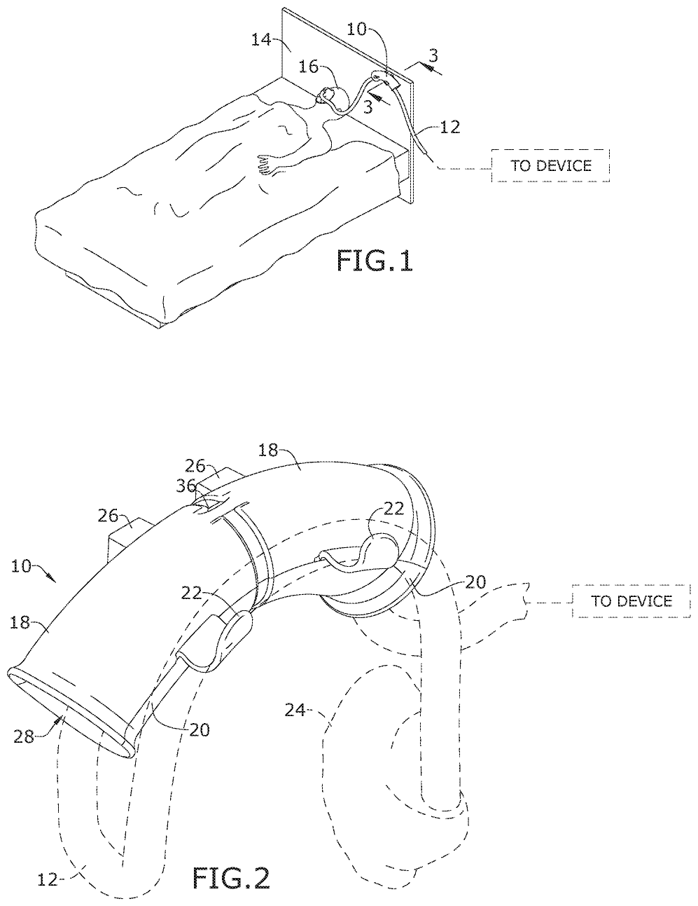

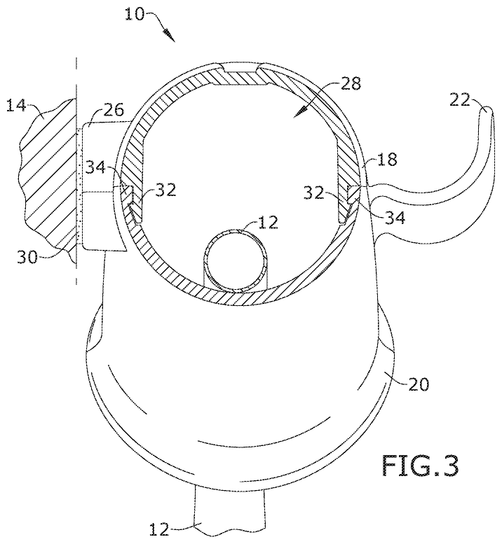

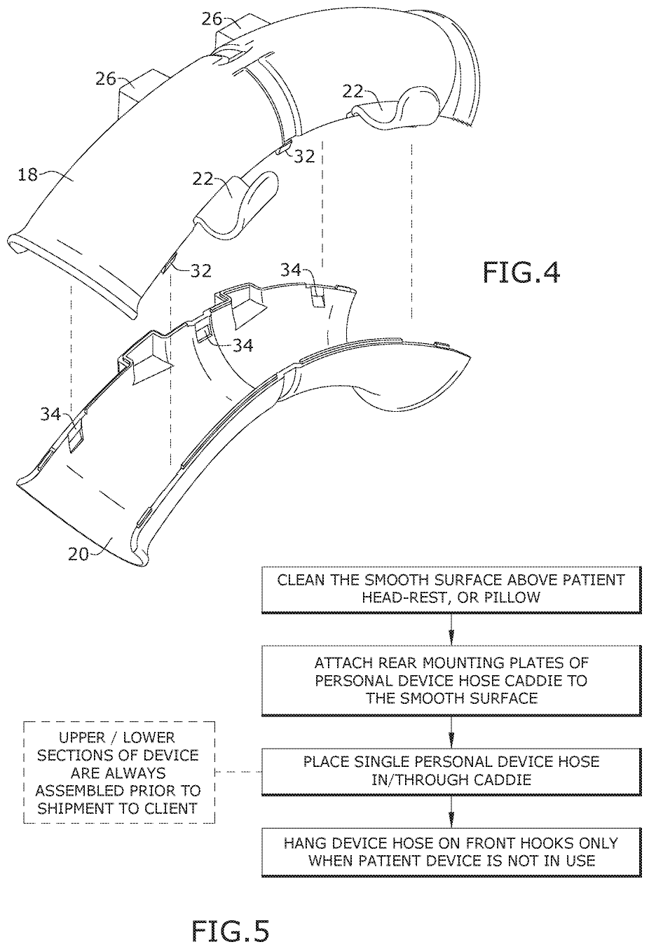

Referring to FIGS. 1 through 5, the present invention includes a Hose Caddy. The Hose Caddy includes a body 10 made of a rigid material. The body 10 includes a tube shape with an outer surface and an inner surface. The inner surface defines a channel 28 having a first open end, and a second open end. A coupler 26 protrudes from a rear side of the outer surface of the body 10 and couples the body 10 to a surface 14. At least one hook 22 protrudes from a front side of the outer surface of the body 10. The present invention isolates the path of air and fluid lines and has no edges that will damage or injure the patient, visitor or caregiver.

In certain embodiments, the body 10 includes a curve defining an arc shape. The body 10 may be about 9 inches in length and about 3.5 inches wide. However, the body 10 may include other dimensions that allow the present invention to work. Front and rear edges defining the first open end and the second open end may flair outwards from a center point of each opening.

In certain embodiments, the body 10 includes an upper half 18 and a lower half 20 each including a half pipe shape. The upper half 18 is releasably coupled to the lower half 20 by male clips 32 and female clips 34. In certain embodiments, the body 10 may further include a circular groove 36 defined along a circumference of the outer surface. The circular groove 36 allows users to attach additional tubes and devices via a zip tie or other cord connector.

The hook 22 may include a first hook 22 and a second hook 22. Each of the first hook 22 and the second hook 22 may protrude from the front side of the body 10. While a patient 16 is not using the medical device, the patient 16 may loop the hose to the first and second hooks 22.

In certain embodiments, the coupler 26 may include a first mount and a second mount. The mounts may be part of the body 10 and extend from the outer surface at the rear side. The mounts may include surfaces planar with one another, and an adhesive 30 disposed on the surfaces. A user may press the mounts and adhesive 30 against the surface 14, thereby adhering the Hose Caddy to the surface 14.

A method of containing a medical hose 12 may include the following steps: provide the Hose Caddy described above; couple the body 10 to a surface 14 above a bed; and run the medical hose 12 from a medical device through the channel 28 of the body 10 and to a patient 16. The step of coupling the body 10 to the surface 14 above the bed may include adhering the body 10 to the surface 14 using an adhesive 30. Once the invention is placed on the selected surface, the patient/caregiver may then use the invention to prevent tangling and separation issues.

It should be understood, of course, that the foregoing relates to exemplary embodiments of the invention and that modifications may be made without departing from the spirit and scope of the invention as set forth in the following claims.

What is claimed is:

1. A hose caddy comprising:
a body made of a rigid material and comprising a tube shape with an outer surface and an inner surface, the inner surface defining a channel having a first open end, and a second open end;
a coupler protruding from a rear side of the outer surface of the body and configured to couple the body to a surface; and
at least one hook protruding from a front side of the outer surface of the body.

2. The hose caddy of claim 1, wherein the ‘at least one hook’ comprises a first hook and a second hook, each comprising a U-shape.

3. The hose caddy of claim 1, wherein the coupler comprises a first mount and a second mount, each comprising surfaces planar with one another, and an adhesive disposed on the surfaces.

4. The hose caddy of claim 1, wherein the body comprises an upper half and a lower half each comprising a half pipe shape, wherein the upper half is coupled to the lower half.

5. The hose caddy of claim 1, further comprising a circular groove defined along a circumference of the outer surface.

6. The hose caddy of claim 1, wherein the body comprises a curve defining an arc shape.

7. A method of containing a hose comprising:
providing a hose caddy comprising a body made of a rigid material and comprising a tube shape with an outer surface and an inner surface, the inner surface defining a channel having a first open end, and a second open end;
coupling the body to a surface above a bed; and
running the hose from a device through the channel of the body and to a patient.

8. The method of claim 7, wherein the step of coupling the body to a surface above the patient rest area comprises adhering the body to the surface using an adhesive.

9. The method of claim 7, wherein the body comprises an upper half and a lower half, each comprising a half pipe shape, wherein the upper half is coupled to the lower half.

10. The method of claim 7, wherein the hose caddy further comprises a hook protruding from a front side of the base.

11. The method of claim 10 further comprising a step of: hanging a portion of the hose from the hook.

12. The method of claim 7, wherein the body comprises a curve defining an arc shape.
